(19) United States
(12) Patent Application Publication
(10) Pub. No.: US 2004/0071733 A1
(43) Pub. Date: Apr. 15, 2004

(54) Baculovirus vector vaccine

(76) Inventors: Hiroshi Takaku, Narashino-shi (JP); Yosuke Suzuki, Tsukuba-shi (JP)

Correspondence Address: Jane Massey Licata, Licata & Tyrrell, 66 East Main Street, Marlton, NJ, 08053, US

(21) Appl. No.: 10/470,888

(22) PCT Filed: Feb. 5, 2002
(86) PCT No.: PCT/JP02/00913

(30) Foreign Application Priority Data
Feb. 5, 2001 (JP) 2001-027648

(51) Int. Cl. A61K 39/12; A61K 39/145

(52) U.S. Cl. 424/204.1; 514/44; 424/206.1

(57) ABSTRACT

It is intended to provide virus vector vaccine preparations containing as the main component a virus vector with the use of a virus showing no pathogenicity on humans and being free from a risk of the re-acquisition of pathogenicity. Namely, vaccine preparations containing as the main component a baculovirus having a gene encoding an antigen integrated thereinto are provided.

### TECHNICAL FIELD

The present invention relates to provision of a vaccine preparation that has no pathogenicity, has no possibility of reacquiring pathogenicity, and is capable of making an antigen act persistently on a living body.

### BACKGROUND ART

Conventionally, most of the virus vectors that have been used for human gene therapy, etc. are derived from viruses such as adenovirus that show pathogenicity in humans, and their attenuated strains, etc., whose pathogenicity is suppressed, are utilized. However, since genes coding for this pathogenicity are not completely removed, a cell mediated immunoreaction in a host excludes all the infected cells from the host, and as a result expression of the gene is only transient. Furthermore, viruses such as adenovirus and vaccinia virus that humans already have immunity to are neutralized at an early stage by neutralizing antibodies. That is, when a virus vector utilizing adenovirus or vaccinia virus is administered continuously, the therapeutic effects are reduced due to the presence of the neutralizing antibody. Furthermore, in retroviruses, etc. there are the problems of activation of a proto-oncogene due to insertion mutation into a host gene, cytotoxicity, etc., and reacquisition of pathogenicity in crossing with a replication competent virus is also a concern.

Since recombinant virus vector vaccines that are infectious to living bodies produce exogenous antigens in infected cells, they bring about the same antigen molecular configuration as that of the original pathogenic antigen, that is, modifications such as authentic processing, sugar chain addition, and phosphorylation. This introduces a more native antigenicity and causes strong immunogenicity. Furthermore, in addition to hormonal immunity, it is expected to induce cytotoxic T lymphocyte (CTL)-mediated cellular immunity. Existing virus vectors currently under development include adenovirus (Lee H K, et al., Cold Spring Harbor Laboratory Press., (1991)) and vaccinia virus (Watanabe K, et al., Vaccine., 7, (1989)). A recombinant vaccinia virus has an exogenous antigen gene inserted into a region not essential for replication of the vaccinia virus, does not replicate but has infectivity, and can be applied as a gene expression vector, as a plasmid in E. coli. However, since the vaccinia virus used for vaccination against smallpox has caused episodes of transient encephalitis, etc., though to a slight extent (Fenner F, et al., World Health Organization, (1988)), a temperature-sensitive attenuated strain has been developed in the same way as a live vaccine for influenza. Furthermore, many of the viruses, including vaccinia virus, that are applied in existing virus vector vaccines show pathogenicity in humans, and it is suggested that, even in the selection of attenuated strains, unstable factors for a reversion to virulence cannot be completely eliminated.

Moreover, since a majority of pathogens carry out attachment and invasion via the skin or a mucous membrane and infect transmucosally via mainly nasal mucosa, laryngopharyngeal mucosa, gastrointestinal mucosa, etc., it is considered to induce a secretory IgA antibody on the mucous membrane surface in order to prevent infection. Induction of such an IgA antibody on the mucous membrane surface is difficult to carry out by subcutaneous vaccination, and it is carried out by a transmucosal vaccine such as nasal or oral immunization (H. Takekawa, et al., ‘Oral Vaccines’, Igaku-no-ayumi (Medical Advances), Vol. 181, No. 9, p. 734, 1997). However, development of conventional vaccines utilizing lymphatic tissue distributed in a mucous system is carried out utilizing human pathogenic viruses such as polio virus, RSV (respiratory syncytial virus), rotavirus, adenovirus, influenza virus, rabies virus, and HIV (human immunodeficiency virus), which potentially have unstable factors for reversion to virulence, etc.

Conventionally, the production of protein is known in which used is baculovirus, whose range of infection hosts includes many arthropods, including insects. JP, A, 7-228595 discloses that Aujeszky's disease virus gII protein is produced using a baculovirus, and that the protein so produced is purified from the baculovirus and used as a vaccine.

In recent years, attention has been focused on gene vaccines (DNA, RNA vaccines) that induce an immunoresponse by administering a gene (DNA, RNA) coding for antigen information directly to living bodies (Ulmer J B, et al., Science., 259, (1993)), the gene vaccines being different from deactivated vaccines or live vaccines, which have problems with respect to clinical efficacy and safety. DNA vaccines can exhibit a higher immune enhancing effect by the use in combination with an effective adjuvant. However, it has been pointed out that the current problems of DNA vaccines relate to the dose and low expression; in order to obtain a sufficient immunoresponse within muscle and skin at least a few hundred micrograms of DNA is considered necessary; and even by employing a physical technique such as a gene gun it is mainly hormonal immunity that is induced, and low induction of cell mediated immunity is achieved.

On the other hand, it has been shown that baculovirus can infect various animal cells, including human liver cells (Hofmann, C., Sandig, V., Jenning, G., Rudolph, M., Schlag, P., and Strauss, M. (1995) “Efficient gene transfer into human hepatocytes by baculovirus vectors” Proc Natl Acad sci USA. 92, 10099-10103, Shoji I., Aizaki H., Tani H., Ishii K., Chiba T., Saito I., Miyamura T., and Masyuura Y. (1997) “Efficient gene transfer into various mammalian cells, including non-hepatic cells, by baculovirus vectors” J. Gen. Virol. 78, 2657-2664, and Boyce, F. M. and Bucher, N. L. R. (1996) “Baculovirus-mediated gene transfer into mammalian cells” Proc Natl Acad sci USA. 93, 2348-2352).

Furthermore, Barsoum, et al. (Barsoum, J. et al., Hum. Gene Ther. 8, 2011-2018 (1997)) has disclosed that transduction into mammalian cells is conducted using a baculovirus expressing the G glucoprotein of vesicular stomatitis virus in its envelope.

However, although the use, as a vaccine, of a protein expressed and purified using baculovirus has been, use of a baculovirus vector itself (virus particles or virus nucleic acid) as a vaccine preparation has not hitherto been demonstrated.

In the case where a conventional virus vector utilizing adenovirus or vaccinia virus is used as a gene vaccine, there is a possibility of reacquisition of virus pathogenicity. Moreover, in general, with regard to viruses that have a high possibility of infecting humans, there has been such a problem many people having infection experience, thus having neutralizing antibodies, and a virus vaccine being not readily introduced due to an immunoreaction.

In order to solve these problems, there has been a desire for a virus vector that utilizes a virus having no pathogenicity in humans and that can be used as a gene vaccine.

### DISCLOSURE OF INVENTION

While carrying out an intensive investigation in order to solve the above-mentioned problems, the present inventors have found that, by using as a virus vector vaccine preparation a virus vector utilizing an insect pathogenic virus, etc. such as baculovirus, for which humans are hosts in its natural state and has no pathogenicity in humans, a vaccine preparation can be provided that can persistently express antigens and is free from reacquiring pathogenicity; and as a result of further investigation, the present invention has been accomplished.

That is, the present invention relates to a vaccine preparation containing as a main component a non-human host virus in which a gene coding for an antigen has been incorporated.

Furthermore, the present invention relates to the above-mentioned vaccine preparation wherein the virus not hosted by humans is a baculovirus.

Moreover, the present invention relates to the above-mentioned vaccine preparation wherein the antigen is a virus membrane protein antigen.

Furthermore, the present invention relates to the above-mentioned vaccine preparation wherein the virus membrane protein antigen is an influenza HA antigen.

Moreover, the present invention relates to the above-mentioned vaccine preparation wherein the preparation further contains an adjuvant and/or a pharmaceutically acceptable carrier.

Furthermore, the present invention relates to the above-mentioned vaccine preparation wherein the preparation is nasally administered.

When a human is infected with a virus that has an infection host range that includes humans in its natural state and is infectious to humans, such as an influenza virus, a neutralizing antibody to the infecting virus is produced. Because of this, if a virus pathogenic to humans such as an influenza virus is used as a virus vector for use in a vaccine preparation, then the effect of the vaccine preparation may be in sufficiently enhanced due to a neutralizing antibody that already exists in the human body.

In contrast, the non-human host virus used in the present invention does not normally infect humans. That is, if a non-human host virus is used as a vaccine preparation, since humans do not have a neutralizing antibody therefor, high efficacy is obtained from the vaccine preparation. The non-human viruses referred to in the present specification mean viruses that do not include as an infection host humans in their natural state, and denote insect viruses, plant viruses, etc. that do not infect humans in their natural state.

In particular, a baculovirus, which is a virus pathogenic to insects and is used suitably in the present invention, does not include humans as its infection host in its natural state. However, gene expression thereof is possible not only in insect cells but also in many mammalian cells including human liver cells and, moreover, by selecting an appropriate promoter high gene expression is possible. Furthermore, a baculovirus has no neutralizing antibody in humans; since it is a nonproliferating virus in mammalian cells, the degree of damage to cells is low, and the use thereof achieves a very useful vaccine preparation.

### BRIEF DESCRIPTION OF THE DRAWINGS

FIG. 1A is a schematic diagram of a baculovirus transfer vector that expresses an HA gene.

FIG. 1B is a schematic diagram showing the structure of an HA antigen-expressing recombinant baculovirus vector.

FIG. 2 is a diagram (photograph) showing the expression of an HA protein in an HA antigen-expressing recombinant baculovirus rBacCAG/HA infected human liver cancer cell strain (Huh-7). (A) shows a fluorescent image, and (B) shows a bright field image.

FIG. 3 is a graph showing body weight changes of mice during an immunization period, the mice having been immunized with an intraperitoneally vaccinated HA antigen-expressing recombinant baculovirus.

FIG. 4 is a graph showing the results of an influenza virus infection protection test in mice immunized with an intraperitoneally vaccinated HA antigen-expressing recombinant baculovirus.

FIG. 5 is a graph showing the results of an influenza virus infection protection test in mice immunized via various administration routes with an HA antigen-expressing recombinant baculovirus.

### BEST MODE FOR CARRYING OUT THE INVENTION

Modes for carrying out the present invention are explained below by reference to examples of virus vector vaccines to which a baculovirus, which is a non-human host virus, is applied.

The vaccine preparation of the present invention is a vaccine preparation containing as a main component baculovirus particles or a baculovirus nucleic acid into which incorporated is a gene that expresses a polypeptide that becomes a desired antigen, and may contain an adjuvant and/or a pharmaceutically acceptable carrier.

In the present invention, any gene may be incorporated into the baculovirus as long as it codes an antigen to which a living body acquires immunity. Such antigens are not particularly limited, but membrane proteins forming the envelope of a pathogenic virus are preferable, for example, since the living body can acquire an antibody to the pathogenic virus. Examples of such virus membrane protein antigens include influenza-derived antigens such as an HA antigen, an NP antigens, an M antigen, an NS1 antigen, and an NA antigen.

The vaccine preparation of the present invention is usually administered without any adjuvant since it is accompanied by a risk of destroying the viral envelope, thereby decreasing the efficiency of entering cells. The adjuvant that can be used in the vaccine preparation of the present invention is not particularly limited as long as it does not destroy the viral envelope.

The pharmaceutically acceptable carrier that can be used in the vaccine preparation of the present invention is not particularly limited as long as it does not destroy the viral envelope, and examples thereof include an amino acid, albumin, and polyethylene glycol.

The method for administering the vaccine preparation of the present invention is not particularly limited, so that it can be administered nasally, muscularly, subcutaneously, peritoneally, etc. In administering the baculovirus vector vaccine preparation of the present invention, it is desirable to administer it so as to avoid the action of a complement in the blood stream, so that nasal administration and peritoneal administration are preferred.

Furthermore, when an influenza-derived antigen is used in the vaccine preparation of the present invention, the method for administering the vaccine preparation is particularly preferably nasal administration.

An influenza virus is primarily an airborne infection, and infects by virus particles adhering to a mucous membrane of an airway such as a nasal cavity. When the influenza virus infects the mucous membrane of the nasal cavity, an antibody, in particular IgA, is produced, which responds effectively to an influenza HA antigen, thus producing an immunoresponse. Taking this into consideration, administration is preferably carried out in line with the route of infection of a pathogenic virus. For example, administration of a vaccine preparation against influenza, which infects via the mucous membrane of the nasal cavity, is preferably carried out nasally using an aerosol preparation, etc.

The present invention is hereinafter explained in further detail by reference to examples, but the present invention is not limited only to the examples below.

### EXAMPLES

### Example 1

### Preparation of an HA Antigen-Expressing Recombinant Baculovirus and Evaluation of Gene Expression in Various Mammalian Cell Strains

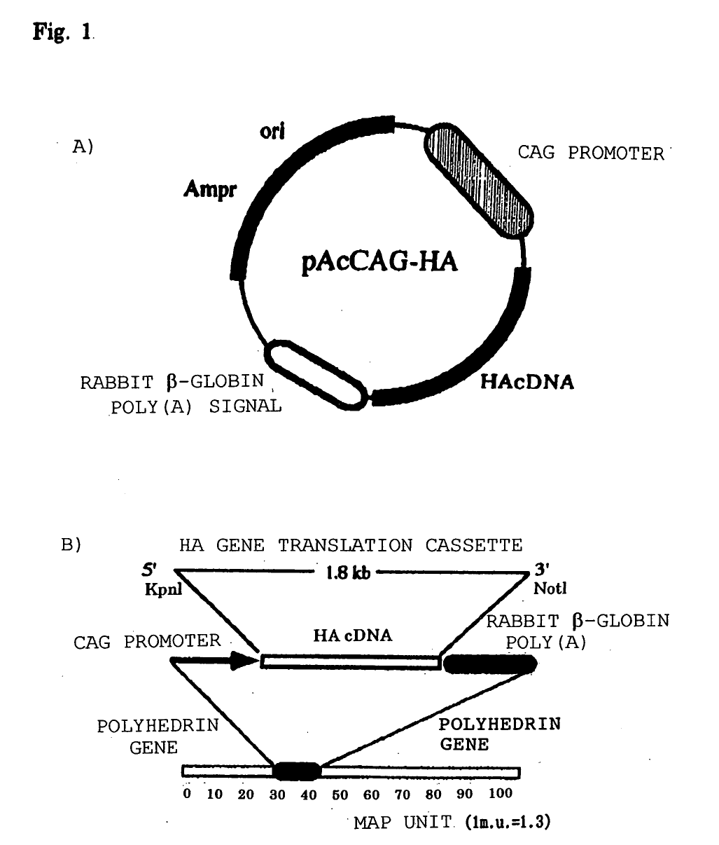

Influenza type A virus (A/PR/8/34: H1N1) HA (hemagglutinin) gene (pJZ102 (HA gene insertion vector) obtained-from Dr. S. Nakata, Screening Laboratory, Tansaku Institute, Yamanouchi Pharmaceutical Co., Ltd.) was incorporated into a pAcCAG baculovirus transfer vector having a CAG promoter (Chicken β-actin-derived promoter)(obtained from Dr. Y. Matsuura, National Institute of Infectious Diseases) (FIG. 1A), and an HA antigen-expressing recombinant baculovirus was prepared by a homologous recombination method in sf-9 cells (FIG. 1B). sf-9 cells are of a Noctuidae larva-derived cell line (Spodoptera frugiperda cells) and are generally used for baculovirus replication. The sf-9 cells used were cells contained in the BaculoGold™ Starter Package manufactured by Pharmingen. The sf-9 cells were cultured in an incubator at 27° C. using 10% inactivated fetal bovine serum (FBS; Sanko Junyaku Co., Ltd.) in an insect cell culture medium (TMN-FH; Pharmingen).

In the figure, ‘Ampr’ denotes an ampicillin resistance gene. ‘CAG promoter’ denotes a Chicken β-actin-derived promoter. ‘Rabbit β-globin poly A signal’ denotes a rabbit β-globin derived translation termination signal. ‘Polyhedrin gene’ denotes a baculovirus gene containing the homologously recombined baculovirus DNA sequence.

The constructed recombinant virus was amplified by 5 passages to sufficiently enhance the virus titer sufficiently, and the recombinant virus was then purified by a 10%/60% sucrose density gradient centrifugal method at 25,000 rpm and 4° C. for 90 minutes.

The titer of the purified recombinant baculovirus was determined by a plaque assay, and subsequently (1) HA gene expression level, (2) cytotoxicity (virulence), and (3) virus titer were evaluated over time in various types of mammalian cell strains.

(1) HA gene expression level

The level of expression of the HA gene in various mammalian cell strains by the constructed, purified recombinant virus rBacCAG/HA was confirmed by a fluorescent antibody technique using FITC-labeled anti-HAMAb (Denka Seiken Co., Ltd.). That is, the purified recombinant baculovirus was made to be adsorbed by and infect cells at an moi of 50 at 37° C. for 2 hours; 24 hours later FITC-labeled anti-HAMAb was reacted therewith, and inspection was carried out using a confocal laser microscope. ‘Moi’ stands for multiplicity of infection, also being referred to as the virus infectivity titer, and is the average number of infectious viruses per cell when cells are infected with the virus.

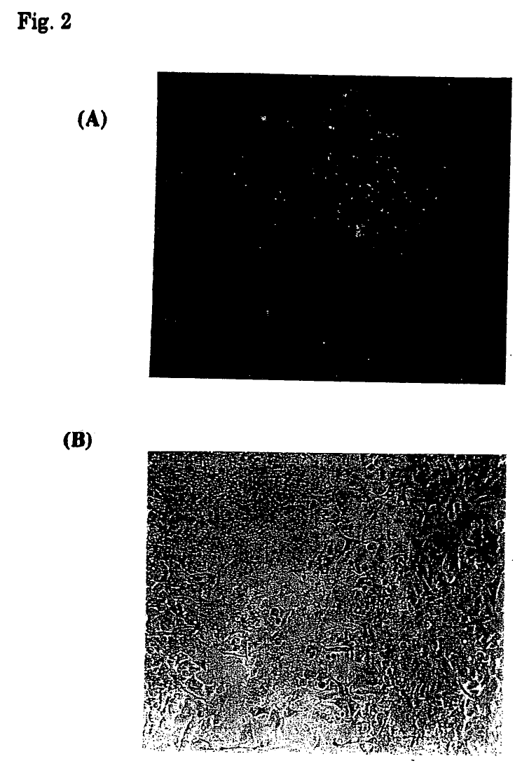

From the inspection, particularly high fluorescent strength was observed for a human liver cell strain (Huh-7), etc. (FIG. 2(A)). FIG. 2(B) shows the result of bright field observation of the human liver cell strain (Huh-7).

(2) Cytotoxicity (virulence)

Evaluation of the cytotoxicity was carried out by culturing various mammalian cell strains in a 96 well cell culture plate with 2×10⁴ cells/well, then inoculating the cells with the recombinant virus rBacCAG/HA at serial dilutions from an moi of 100, adding an MTT reagent after 1, 3, and 5 days, measuring the absorbance at 540/690 nm using an absorbance microplate reader, and calculating the viability.

The mammalian cell strains tested were HeLa (human endocervical carcinoma derived; obtained from Riken Gene Bank), MDCK (dog renal cortex derived; obtained from Riken Gene Bank), HepG2 (human hepatocellular carcinoma derived; obtained from Riken Gene Bank), Huh-7 (human hepatocellular carcinoma derived; obtained from Riken Gene Bank), CPK (pig renal cell derived; obtained from Riken Gene Bank), C127 (mouse thymic epithelial carcinoma cell derived; obtained from Dr. S. Nakata, Screening Laboratory, Tansaku Institute., Yamanouchi Pharmaceutical Co., Ltd.), Cos-7 (monkey renal cell derived; obtained from Riken Gene Bank), WI-38 (human normal lung tissue derived; obtained from Riken Gene Bank), and 293 cells (human renal cell derived; obtained from Riken Gene Bank).

The moi-dependent cytotoxicity of the constructed, purified recombinant baculovirus rBacCAG/HA was examined in various mammalian cell strains.

Table 1 shows the results of evaluation of the cytotoxicity when a human liver cell strain (Huh-7) and insect cells (sf-9) were infected with the HA antigen-expressing recombinant baculovirus.

Table 1 Evaluation of cytotoxicity when human liver cell strain (Huh-7) and insect cells (sf-9) were infected with HA antigen-expressing recombinant baculovirus

(Virus

| | |
|---|---|
| infectivity | Cell viability (%) |

| titer) | | 0.01 | 0.1 | 1 | 5 | 10 | 20 | 50 | 100 |
|---|---|---|---|---|---|---|---|---|---|
| sf-9 | 1 | 100.0 | 96.4 | 100.0 | 90.1 | 85.1 | 81.6 | 81.2 | 72.4 |
| | 3 | 84.5 | 63.4 | 51.7 | 40.3 | 38.4 | 37.2 | 33.8 | 32.5 |
| | 5 | 63.0 | 48.4 | 29.1 | 20.5 | 15.3 | 12.2 | 12.5 | 11.6 |
| Huh-7 | 1 | 100.0 | 100.0 | 100.0 | 100.0 | 100.0 | 100.0 | 100.0 | 93.3 |
| | 3 | 100.0 | 100.0 | 100.0 | 100.0 | 100.0 | 100.0 | 100.0 | 81.7 |
| | 5 | 97.2 | 100.0 | 100.0 | 95.5 | 89.6 | 94.4 | 95.8 | 100.0 |

After infectivity titer-dependently infecting sf-9 and Huh-7 cell strains with the HA antigen-expressing recombinant baculovirus, the cell viability was measured by the MTT method-after 1, 3, and 5 days.

The cell viability of sf-9, which is a baculovirus host cell, was 11.6% for an moi of 100 after 5 days, whereas the cell viability of the human liver cell strain (Huh-7) was 100% for an moi of 100 after 5 days, and there was no apparent cytotoxicity (Table 1).

Table 2 shows the results of evaluation of the cytotoxicity when various mammalian cell strains were, infected with the HA antigen-expressing recombinant baculovirus.

Table 2 Evaluation of cytotoxicity when various mammalian cell strains were infected with HA antigen-expressing recombinant baculovirus

Cell viability (%)

| (Virus infectivity titer) | 10 | 50 | 100 |
|---|---|---|---|
| sf-9 | 38.4 | 33.8 | 32.5 |
| Huh-7 | 100.0 | 100.0 | 99.9 |
| HepG2 | 99.0 | 95.9 | 95.9 |
| HeLa | 98.2 100.0 | 100.0 | |
| MDCK | 88.1 | 95.0 | 98.0 |
| C127 | 87.5 | 76.8 | 77.6 |
| Cos-7 | 94.3 | 89.6 | 92.5 |
| WI-38 | 89.3 | 89.3 | 83.9 |

After infecting each cell strain with the HA antigen-expressing recombinant baculovirus with infectivity titers of 10, 50, and 100, the cell viability was measured by the MTT method-after 3 days.

No cytopathogenic effects were observed, not only for Huh-7 but also for human derived cell strains (HeLa, HepG2, WI-38, 293) and the other animal derived cell strains (Cos-7, MDCK, C127, CPK) (Table 2). In a control test, which was the same cytotoxicity test carried out for sf-9 cells, which were the original host cells, it was observed that the cytotoxicity increased as the virus infectivity titer increased. The cell viability after 5 days with an moi of 100 was about 12% (Table 1).

### Example 2

### Measurement of Anti-HA Antibody Titer in Mice Immunized with an HA Antigen-Expressing Recombinant Baculovirus rBacCAG/HA

The HA antigen-expressing recombinant baculovirus rBacCAG/HA was administered intraperitoneally (i.p.) to 6 week old female BALB/c mice in an unanesthetized state at doses of 1×10⁵, 10⁶, 10⁷, and 10⁸ pfu/mouse. After 2 weeks, boosters were administered using the same doses. Blood was collected 1 and 3 weeks after the secondary inoculation, the collected serum samples were diluted 100 times with PBS(−), and the anti-HA antibody titer was measured by the ELISA method.

Specifically, after the type A influenza virus HA antigen was solidphased in an ELISA 96 well plate at a concentration of 7.5 μg/ml at 4° C. for 18 hours, incubation was conducted at 37° C. for 2 hours after addition of a standard control HA antibody (10, 20, 40, 80, 160, 320 ng/ml) and an immune serum sample. Subsequently, the mixture was reacted with a biotin-labeled anti-mouse IgG (γ) antibody (1 μg/ml) and an alkaline phosphatase-labeled streptavidin (0.2 μg/ml), pNPP was finally added thereto, and the absorbance at 405 nm was measured using an absorbance microplate reader to calculate the anti-HA antibody titer.

The results are given in Table 3.

Table 3 Anti-HA antibody titer in mice immunized with intraperitoneally vaccinated HA antigen-expressing recombinant baculovirus

| | Serum IgG titer | | ELISA (μg/ml) | |
|---|---|---|---|---|
| | 1.08 × 10^{5a} | 1.08 × 10⁶ | 1.08 × 10⁷ | 1.08 × 10⁸ |
| After 3 weeks ^{b, c} | 2.6 ± 0.2 | 3.3 ± 0.6 | 4.2 ± 0.2 | 6.1 ± 0.7 |
| After 5 weeks | 2.8 ± 0.4 | 4.7 ± 0.4 | 6.0 ± 1.5 | 5.9 ± 1.3 |

a Mice were intraperitoneally inoculated with 10⁵, 10⁶, 10⁷ and 10⁸ pfu of the HA antigen-expressing recombinant baculovirus, and 2 weeks later boosters were administered using the same doses.

b, c The immune serum samples were collected 1 and 3 weeks after the boosters (3 and 5 weeks after the primary inoculation).

From the results in Table 3, it was found that the serum samples after 3 and 5 weeks showed increased anti-HA antibody titer dependent on the recombinant virus inoculum dose. The anti-HA antibody titer for each dose was: Week 3; 2.6±0.2 for 1×10⁵ pfu/mouse, 3.3±0.6 for 1×10⁶ pfu/mouse, 4.2±0.2 for 1×10⁷ pfu/mouse, and 6.1±0.7 μg/ml for 1×10⁸ pfu/mouse, and Week 5; 2.8±0.4 for 1×10⁵ pfu/mouse, 4.7±0.4 for 1×10⁶ pfu/mouse, 9.7±7.6 for 1×10⁷ pfu/mouse, and 10.1±2.6 μg/ml for 1×10⁸ pfu/mouse.

Furthermore, immunization with the HA antigen-expressing recombinant baculovirus rBacCAG/HA was carried out under the same test schedule through other administration routes such as intradermal (i.d.), intramuscular (i.m.), and intranasal (i.n.) routes; and the anti-HA antibody titer 3 weeks after the primary inoculation (1 week after the secondary inoculation) was calculated by the ELISA method. The results are given in Table 4. The survival rate in Table 4 will be explained in Example 3.

Table 4 Anti-HA antibody titer in mice immunized via various administration routes with HA antigen-expressing recombinant baculovirus rBacCAG/HA and protection effects against influenza virus infection

| | Serum IgG titer | ELISA (μg/ml) | Survival |
|---|---|---|---|
| | After 3 weeks ^{b, c} | After 5 weeks | rate (%) |
| Intramuscular (i.m.) ^{a} | 7.0 ± 0.6 | — | 38 |
| Intranasal (i.n.) | 7.3 ± 2.3 | 8.4 ± 1.5 | 100 |
| Intraperitoneal (i.p.) | 6.1 ± 0.7 | 5.9 ± 1.3 | 50 |
| Intradermal (i.d.) | 4.1 ± 0.4 | 3.4 ± 0.2 | 0 |

a Mice were inoculated with 1×10⁸ pfu of the HA antigen-expressing recombinant baculovirus intramuscularly (i.m.), intranasally (i.n.), intraperitoneally (i.p.), and intradermally (i.d.), and after 2 weeks boosters were administered using the same doses.

b, c The immune serum samples were collected 1 and 3 weeks after the boosters (3 and 5 weeks after the primary inoculation).

The results were that the anti-HA antibody titers for the various administration routes were 4.1±0.4 for intradermal (i.d.), 7.0±0.6 for intramuscular (i.m.), and 7.3±2.3 μg/ml for intranasal (i.n.) (Table 4).

### Example 3

### Protective Effect against Influenza Virus Infection in Mice Immunized with HA Antigen-Expressing Recombinant Baculovirus rBacCAG/HA

The HA antigen-expressing recombinant baculovirus rBacCAG/HA was administered intraperitoneally (i.p.) to 6 week old female BALB/c mice in an unanesthetized state at doses of 1×10⁷ and 10⁸ pfu/mouse, and 2 weeks later boosters were administered using the same doses. Three weeks after the secondary inoculation, the mice were inoculated intranasally with 100LD50 influenza virus (A/PR/8/34:H1N1) in a Nembutal-anesthetized state. After infection with the influenza virus, a morphological inspection and the body weight change of the mice were recorded daily, and the survival rate up to 14 days later was assessed.

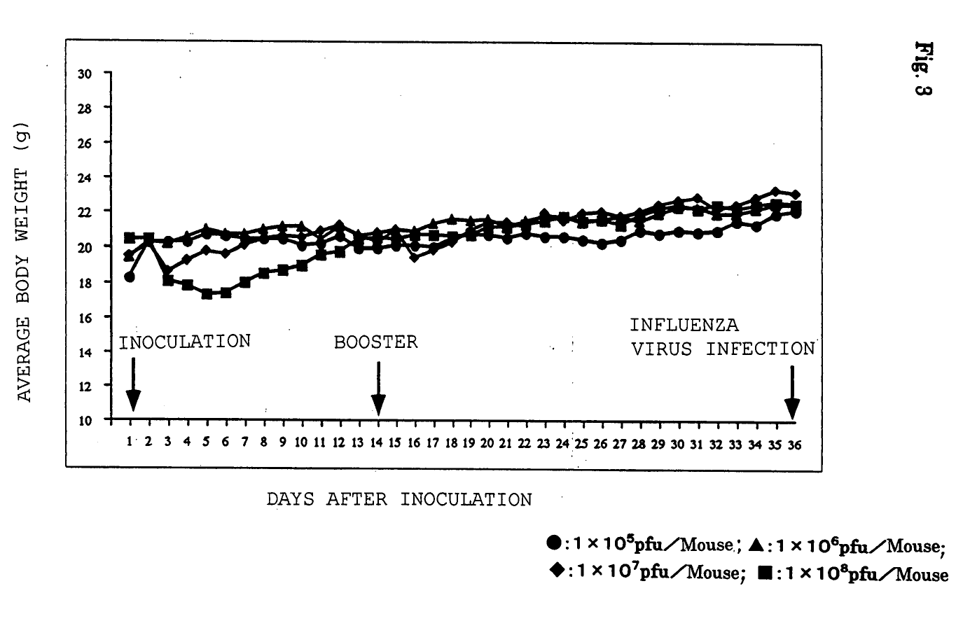

The morphological inspection and the body weight change of the mice were measured daily from the primary inoculation to the influenza virus infection protection test, and the influence on individual mice of the HA antigen-expressing recombinant baculovirus rBacCAG/HA was examined (FIG. 3).

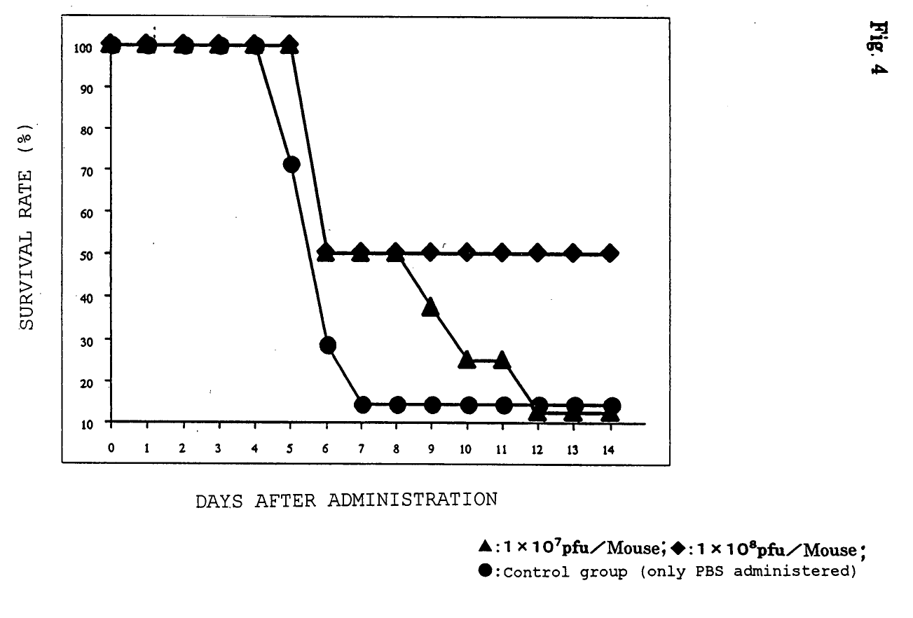

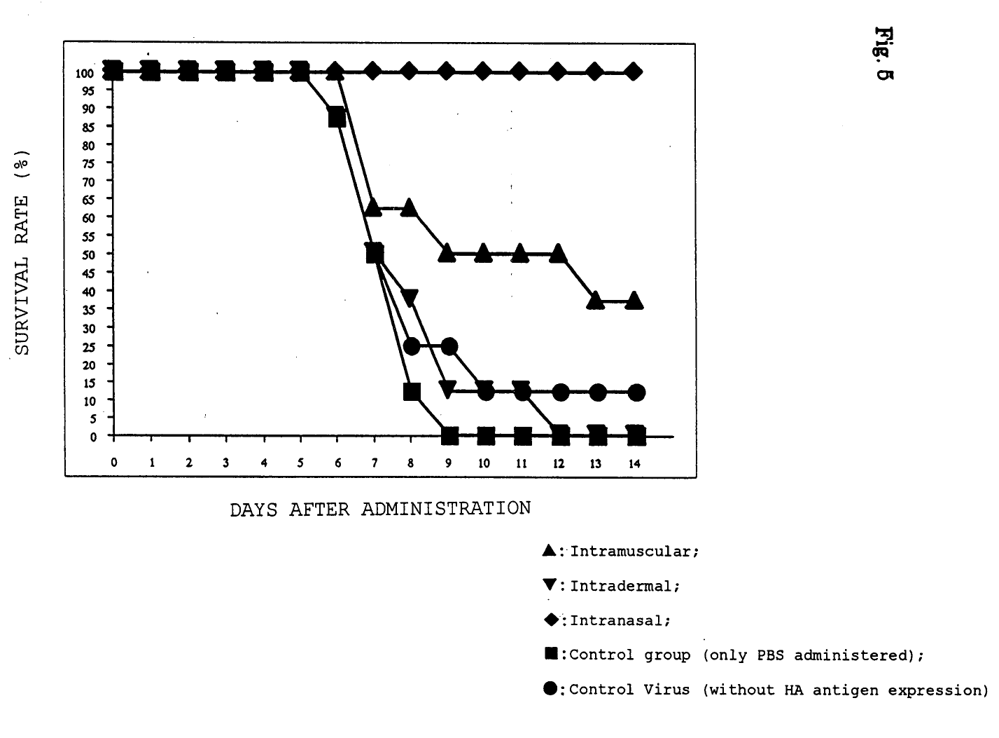

In the virus infection protection test carried out for the immune mice induced with a high antibody titer by infecting intranasally with a lethal dose of influenza virus (A/PR/8/34: H1N1), the number of deaths increased for all the groups around 5 days after the virus infection, 90% of the nonimmune group being dead on the 7th day. On the other hand, among mice immunized with the HA antigen-expressing recombinant baculovirus the survival rate of 50% was observed for the group to which 10⁸ pfu had been administered (FIG. 4). Furthermore, immunization with the HA antigen-expressing recombinant baculovirus rBacCAG/HA was carried out through intradermal (i.d.), intramuscular (i.m.), and intranasal (i.n.) routes, to evaluate the influenza virus infection protection effects. The results were that the mouse survival rate 14 days after immunization through each of the administration routes was 0% for intradermal-(i.d.), 38% for intramuscular (i.m.), and 100% for intranasal. (i.n.) (FIG. 5 and Table 4). The nonimmune group and the control virus inoculation immune group showed a low survival rate (13% for the group to which PBS (−) had been administered, 0% for the control virus inoculation immune group), thus demonstrating the significance of the intranasal immunization with the HA antigen-expressing recombinant baculovirus rBacCAG/HA.

### INDUSTRIAL APPLICABILITY

In accordance with the present invention, a virus vector vaccine preparation can be provided that contains as a main component a virus vector utilizing a virus having no pathogenicity in humans, and that has no possibility of reacquiring pathogenicity.

1. A vaccine preparation containing as a main component a non-human host virus wherein a gene coding for an antigen has been incorporated.

2. The vaccine preparation according to claim 1 wherein the non-human host virus is a baculovirus.

3. The vaccine preparation according to claim 1 or 2 wherein the antigen is a virus membrane protein antigen.

4. The vaccine preparation according to claim 3 wherein the virus membrane protein antigen is an influenza HA antigen.

5. The vaccine preparation according to any one of claims 1 to 4 wherein the preparation further contains an adjuvant and/or a pharmaceutically acceptable carrier.

6. The vaccine preparation according to any one of claims 1 to 5 wherein the preparation is nasally administered.
